(19) United States
(12) Patent Application Publication
(10) Pub. No.: US 2003/0182696 A1
(43) Pub. Date: Sep. 25, 2003

(54) Method for producing somatic embryos of pine trees (genus pinus)

(76) Inventor: Carlos Ramirez Serrano, Jalisco (MX)

Correspondence Address: Davis Wright Tremaine, 2600 Century Square, 1501 Fourth Avenue, Seattle, WA, 98101, US

(21) Appl. No.: 10/203,062

(22) PCT Filed: Feb. 2, 2001
(86) PCT No.: PCT/MX01/00003

(30) Foreign Application Priority Data
Feb. 3, 2000 (MX) 001185

(51) Int. Cl. C12N 5/04; A01H 7/00; C12N 5/10

(52) U.S. Cl. 800/319; 435/419

(57) ABSTRACT

The invention consists of a new method for obtaining gymnosperm mature somatic embryos, based on chilling treatment and suspension cultures to preserve the embryogenic capacity of immature somatic embryos. The method consists in applying to immature somatic embryos a chilling preservation in liquid medium treatment for as long as a year; afterwards, reinitiation and proliferation are carried out, as well as reduction of proliferation rates, in order to optimize response to maturation promoters, and finally, the maturation of developing somatic embryos. With the exception of the last step, all of the process is carried out through suspension cultures. Embryo germination and plant development were performed by conventional techniques.

### FIELD OF THE INVENTION

The invention is a new alternative method for the preservation of conifer embryogenic tissue during long periods without cryopreservation (−196° C.). For the purpose of evaluating genotypes, it is important to have a low cost protocol for maintaining the embryogenic capacity of immature embryos for a long period. Cryopreservation protocols are high cost methods and they can also generate somaclonal variation. Moreover, it is known that suspension cultures cannot be cryopreserved. The method described here may be used in breeding programs, genetic transformation or whatever activity that needs the conservation of the quality of embryogenic tissue for long periods, including de possibility of reactivating the immature embryos' ability to multiply and mature into plants.

### BACKGROUND OF THE INVENTION

Conifer propagation by in vitro cultures is increasing its importance in order to provide enough material for the demanding of forest products. The seedling propagation is the traditional way to produce large stock of plants for reforestation, mainly for conifer trees. However, intrinsic restrictions avoid the use of sexual reproduction as large scale of seed production and best quality seedlings. Which have caused extensive interest to developed and improve methods for asexual reproduction; especially economically important conifers such Pinus, Pseudotsuga and Picea. Asexual propagation through selection techniques provide high genetic gains by cloning desirable progeny to perform homogeneity and superior growth. Such type of plants is used for reforestation purposes.

Propagation by somatic embryogenesis means the methods whereby embryos are produced in vitro from plant tissue or single cells. The embryos are called as somatic embryos because they are ensuing from vegetative plant tissue rather than from sexual reproduction. Somatic embryogenesis propagation allows to capture all genetic gains and large-scale plant production (Gupta et al., 1993). Furthermore, somaclonal variation (done by in vitro cultures) among subclones belonging to one genotype was not significant, which means that massive propagation of a desirable genotype can be carried out with high genetic stability (Eastman et al., 1991). Conifer somatic embryogenesis is also used to produce large amount of synthetic seeds for low seed production species; for cloning varieties resistant to pesticides, diseases and environmental stress; it is also considered an alternative method for endangered and rare species conservation and also for propagation of ornamental varieties (Attree and Fowke, 1993).

For a long 15 years the conifer regeneration protocols have been improved since the first report of these type of plants were reported by Hakman and von Arnold (1985). However, only few protocols assure massive propagation. The most economically important conifer species are Picea and Pinus. There are 30 Picea species mainly distributed to cooler areas of boreal hemisphere. Pine trees belong to the most important conifer genera with around 140 species scattered around the northern hemisphere, whereas one of the pine diversity region is Mexico with around 60 species (McVaugh, 1992).

The result achieved on conifer somatic embryogenesis shows remarkable difference between Picea and Pinus species from initiation or induction of embryogenic tissue to regeneration of plants. Besides wide range of pine species has been consistently recalcitrant. Among Picea species the initiation rates are around 95% form immature zygotic embryos, and 55% from mature zygotic embryos (Tautorus et al., 1991). It is known that media compound is decisive, such the relation of inorganic nitrogen sources and the type of carbon source are the key for high initiation rates of somatic embryos (von Arnold, 1987). More over, there are quite numerous reports over high yield of mature somatic embryos, even produced by bioreactor process (Attree et al., 1994); however, only few reports have shown the plant regeneration and even less reports the adaptation in nursery conditions. In 1990 Webster and coworkers, have established more than 80% of somatic embryo plants under nursery condition belonging to 71 genotypes. Resently, Hogberg et al., (1988) reported the adaptation of 25% of 2519 mature somatic embryos of Picea abies originated from 12 families. There are successful regeneration reports over of Picea species, such protocols are used for mass propagation of selected material.

Actually the improvement achieved on pine somatic embryogenesis have shown equal or superior results as Picea and Pseudotsuga reports. Early the response to regeneration protocols have had limited response of few genotypes. Gupta y Durzan (1987a) reported regeneration of Pinus taeda, however it was merely a single plant (according to Pullman and Gupta, 1991). Klimaszewska and Smith (1997) have reported the success of regeneration of Pinus strobus somatic embryos, there were no given data of the number of them. Recently, Garin and collaborators (1998) have shown the response of 52 genotypes belonging to 13 families of the same specie, 800 mature somatic embryos from 30 genotypes in 12 families were obtained, with a conversion into plants of only 31%. Lelu et al., (1999) have been shown the achievement of regeneration on Pinus sylvestris and Pinus pinaster; 48% of 360 mature somatic embryos from three genotypes, and 29% of 142 mature somatic embryos from three genotypes, respectively. To date the only method for pine trees that assure the regeneration of a wide range of genotypes and families was reported by Ramirez-Serrano and collaborators (Ramirez-Serrano et al., 1999a, 1999b), through which the production of thousand of somatic embryos from 70 out of 82 genotypes belonging to 19 out of 20 families, were obtained. These results mean that the pine somatic embryogenesis technology can be used for pine breeding and large scale propagation. The full process was done on modified solid medium with various ammonium to nitrate molar ratios depending on the step.

Cryopreservation methods are the most useful tool for preserving embryos or kinds of cells, including animal structures. However, it has been proved that crypreservation of conifer somatic embryos produces somaclonal variation and, consequently, the best genotypes could be altered in the maturation and germination process, although variation was only detected on embryogenic cultures, with no effect on regenerated plants (De Verno et al., 1999). In addition, protoplast derived Picea glauca plants, such protoplast were obtained from cryopreserved embryos, that demonstrate the totipotency of plant cells was preserved (Attree et al., 1989). An other way to maintain the embryogenic capacity for one year is to put small pieces of embryogenic tissue on solid medium onto Erlenmeyer flasks covered by cerum caps (Joy et al., 1991).

Suspension cultures in liquid medium for Picea and Pseudotsuga are efficient routines for both species. The somatic embryos proliferate faster, and costs are lower (Aitken-Christie and Connett, 1992; Gupta et al, 1993). However, most of pine species are recalcitrant to suspension cultures (Handley III, 1996). There are few references on pine suspension cultures: Pinus taeda (Gupta and Durzan, 1987a, 1987b; Gupta and Pullman, 1991; Pullman and Gupta, 1991), Pinus strobus (Finer et al. 1989), Pinus caribaea (Laine and David, 1990; Laine et al. 1992) and Pinus maximartinezii (Ramírez-Serrano 1996). Over the last specie was reported 2.25% of embryogenic tissue induced on immature gametophytes, in a total of 18 genotypes, where only 8 genotypes were directly established in liquid medium (without treatment in solid medium), proliferated from 50 to 700-1500 immature embryos per ml, in 7 or 15 days between subcultures, depending on the genotype. However, only aberrant embryos were produced (Ramírez Serrano, 1996).

The maturation process is usually started by activated charcoal pretreatment in order to enhance response to maturation medium, by absorbing inhibitors in the medium, such as ethylene and plant growth regulators (George, 1993). The basal solidified medium used is the same as in initiation and proliferation steps, supplemented with a carbon source, a razemic abscisic acid (ABA), and a desiccant compound, such polietilen glicol (PEG) or high sugar's concentration in order to increase osmotic potential (Attree and Fowke, 1993). Dunstan and collaborators (1993) recommend ABA that synchronize the embryo development and increase the rates of mature somatic embryos, as well germination. Another compound that enhances pine somatic embryo's maturation is gellan gum at 1% in the maturation medium, without PEG (Klimazsewska and Smith, 1997; Lelu et al., 1999).

According with the results over regeneration by suspension cultures, there are two pine species: Pinus caribaea (Laine et al., 1992). and Pinus taeda (Handley III, 1996) have reached regeneration and plant development. Merely reports over regenerating plants from either cryopreserved genotypes or non cryopreserved are known. Besides, the major difference with those reports and this method is the employment of chilled immature embryos to achieve mature somatic embryos. Such immature embryos were chilled for a long a year.

Before this method the following patents are hereby incorporated by reference: U.S. Pat. No. 5,491,090 patent explain the difficulties to achieve the establishment by suspension cultures of pine somatic embryos, demonstrating the effectiveness of activated charcoal for the maintenance in liquid cultures of a wide range of genotypes, until plant regeneration. Even so, the preservation procedure of genotypes was other than chilling treatment. The U.S. Pat. No. 0,553,4434 patent protect a new media compounds specific for suspension cultures of Pinus taeda, whereas the nitrogen molar ratios are different to this proposal. The U.S. Pat. No. 0,556,3061 restrict maltose utilization for proliferation cultures on solid medium only; in the other hand, to this method the proliferation is done by suspension cultures, besides the success of this step is other than maltose compound. Over the maturation media composition for conifer somatic embryos, the following patents limit the key compound or mixtures: U.S. Pat. No. 0,503,4326 patent restrict the mixture of ABA and AC in the maturation medium. U.S. Pat. No. 0,503,6007 patent constrain the mixture of PEG, ABA and AC, to enhance maturation response by gradual attenuation of ABA concentration. U.S. Pat. No. 0,518,7092 patent limit the mixture of ABA and carbon source. U.S. Pat. No. 0,523,6841 patent protect the gradual ABA diminution and the increment of desiccant compound. U.S. Pat. No. 0,529,4549 patent restrict the mixture of ABA, AC and giberelic acid. U.S. Pat. No. 0,541,3930 patent protect the mixture of ABA, gelling agent and carbon source. U.S. Pat. No. 0,573,1203 patent take care of the combination of gelling agent, carbon source and ABA. U.S. Pat. No. 0,573,1204 patent restrict the mixture of PEG, AC, ABA and carbon source. U.S. Pat. No. 0,585,6191 patent constrain the mixture of ABA, gelling agent and carbon source. S05985667 patent protect the mixture of ABA, PEG and carbon source. WO9963805A2 patent take care of the increasing levels of plant growth regulators (ABA) and/or desiccant compound.

There are some differences between the patents mentioned and this method in order to produce mature somatic embryos. The immature somatic embryos utilized were from the best genotypes tested in solid media, that means such genotypes produce high yield of mature somatic after establishment in solid medium; chilling treatment was done for a long a year in order to preserve the embryogenic capacity. The maintenance was given by new liquid culture strategy to each genotype for long periods, that means the simultaneous utilization-non alternative, of two mediums, such mediums are modified with different ammonium to nitrate molar ratio and/or low mixture of plant growth regulators; induction of low proliferation rates in order to enhance somatic embryo development by low ammonium to nitrate molar ratio in liquid medium; also was done a treatment in order to block the proliferation with high level of nitrate and activated charcoal in order to augment the response to maturation medium. Also the maturation medium was improved by low ammonium to nitrate (10:90), with high level of gelling agent and lacking of PEG.

The main object is to achieve a pine regeneration method, by utilizing a system other than cryopreservation to maintan the embryogenic capacity of immature somatic embryos for long periods.

Another object is to provide a plant regeneration method of pine species by suspension cultures as previous requirement for genetic transformation by biobalistic protocols.

A further object is to develop a method for regenerating a wide range of genotypes and families, and to assure thousand of mature somatic embryos established in soil.

### SUMMARY OF THE INVENTION

According to the present invention, a new method for preserving embryogenic capacity by chilling suspension cultures has been developed. This method allows the production of gymnosperm mature somatic embryos, starting with chilled immature somatic embryos. The method is characterized by applying to immature somatic embryo from suspension cultures a chilling treatment at 4° C. from 1 to 11 months. Later, preserved immature embryos have to reinitiate proliferation in suspension cultures using the lowest plant growth regulators concentration tested. Establishment and proliferation have been achieved by continuing basal media modification. Prior to maturation treatment, proliferating somatic embryos have to stay in low proliferation liquid medium with ammonium/nitrate molar ratio 10:90 and easy uptake carbon source. Finally, the immature embryos were exposed to pre maturation mediwm supplemented with an adsorbent compound in order to initiate the development of somatic embryos, followed by treatment in maturation medium and supplemented with high nitrate concentration, easy uptake carbon source, maturation promoter and non reaction desiccant compound.

The method is characterized by the preservation of gymnosperm somatic embryos in liquid medium at 4° C. During this step a liquid medium lacking of plant growth regulators, plus easy uptake carbon source, and two nitrogen organic sources was used. This technique allows preservation, for as long as one year, of the embryogenic capacity of immature somatic embryos. Therefore, the present invention utilizes the chilling treatment as an alternative for maintaining the embryogenic capacity of immature somatic embryos without further special requirements, like cryopreservation techniques and equipment.

The next stage is reinitiation of embryogenic tissue after maintenance at 4° C., the time needed by somatic embryos to start again the proliferation in suspension cultures in medium with low concentration of plant growth regulators, which was one of the keys requirements for maintaining adequate proliferation of immature somatic embryos for long periods.

The following step is establishment and continued proliferation of immature somatic embryos, which is to maintain the proper multiplication in suspension cultures all the genotypes that were preserved at 4° C. This is done by utilizing the minimum required amount of immature somatic embryos and the lowest concentration of plant growth regulators tested. This combination allowed a stable proliferation without immature embryo morphological changes for more than a year. Ammonium/nitrate molar ratios were modified as needed in medium composition.

The method comprises also a reduction of immature somatic embryo proliferation in suspension cultures, in order to improve response during maturation process by subculturing in a medium supplemented with low ammonium to nitrate molar ratio and lacking of plant growth regulators. Under this treatment it is assumed that in immature embryos the nitrogen pathway changes in order to utilize nitrate instead of ammonium decreasing proliferation thereby encouraging a better maturation promoter action.

The method also includes the maturation process starting with somatic embryo development. Embryogenic masses have to be washed at least three times before being transferred to filter paper. After that, the washed embryos have to be transferred to medium with adsorbent compound, ammonium to nitrate 10:90 molar ratio, a carbon source and no plant growth regulators. Required treatment time depends on the enlargement of suspensor cell and on increase of volume of embryo head, which is the signal for transferring developing embryos onto maturation medium. The maturation medium is characterized by a low ammonium to nitrate (10:90 ratio), a carbon source, a maturation promoter and a desiccating compound. Maturation treatment depends on the time in which somatic embryos develop cotyledons and are ready for the dormancy period (not included in this method). Gymnosperm somatic embryos produced by the present invention include conifer somatic embryos.

The present invention has the advantage of maintaining embryogenic capacity of valuable genotypes by simple and cost-effective technique, at least for the time needed for evaluation, it does not require dangerous substances and high tech equipment.

The present invention is a significant advance in conifer somatic embryogenesis research, especially for the Pinaceae family, because it is now possible to preserve a wide range of genotypes, including cryopreserved genotypes. We have been able to determine that each genotype needs different time to start proliferation. Now it is possible to test a wide range of genotypes in order to evaluate their capacity in ex vitro conditions. Also, the response to biolistic genetic transformation protocols can be evaluated, without using high cost tech and sophisticated equipment.

### BRIEF DESCRIPTION OF THE FIGURES

FIG. 1a shows how to preserve immature somatic embryos by chilling at 4° C. in liquid medium under sterile conditions.

FIG. 1b shows the influence of immature somatic embryo conservation time (CT) over reinitiation time (RT) after chilling at 4° C.

FIG. 2a shows the subculture effect on the number of somatic embryos/ml (E) that proliferate by the influence of different plant growth regulator concentration (GR).

FIG. 2b shows the manner in which the way to reduce proliferation from all genotypes by using different ammonium to nitrate molar ratio was established. The lowest proliferation is obtained by a treatment with ammonium to nitrate ratio of 10:90.

FIG. 3a shows the high amount of immature somatic embryos in suspension cultures. This amount depends on the genotype. The best proliferation was achieved in a medium supplemented with 0.5 mg/l2,4-D and 0.25 mg/l BA.

FIG. 3b shows an immature somatic embryo in proliferation liquid medium, supplemented with 0.5 mg/l2,4-D and 0.25 mg/l BA.

FIG. 4a shows high occurrence of mature somatic embryos treated with maturation medium supplemented with ammonium to nitrate ratio of 10:90, 3% maltose, 80 μM ABA, and 0.55% gellan gum.

FIG. 4b shows a mature somatic embryo treated with the maturation medium.

FIG. 4c shows the root development of pine emblings.

### DETAILED DESCRIPTION OF THE INVENTION

According with the present invention, a new method for gymnosperm plant regeneration has been developed. The key step is chilling the immature somatic embryos at 4° C. from both cryopreserved or no cryopreserved genotypes, into any known liquid medium used for conifer somatic embryogenesis, which allows the preservation of embryos for approximately one year. Suspension cultures were used in all stages of the method, with the exception of maturation process. The method includes reinitiation of proliferation of embryogenic tissue from 1 to 3 months, establishment and continuous proliferation of immature embryos, reduction of embryo proliferation in order to improve maturation response; the maturation process including the starting of somatic embryo development, and their maturation in solid medium. Depending on the step of the process, liquid culture media were supplemented with special ammonium to nitrate molar ratios, with or without plant growth regulators and easy uptake carbon source.

This invention requires understanding and control of certain biological factors that have an effect on latency, induction, proliferation and maturation of somatic embryos, since it is known that stages of embryo development are similar in both zygotic and somatic embryos. The effect of concentration of plant growth regulators on proliferation is also important, since it forces each somatic embryo to generate a new one through cleavage, as well as the ammonium to nitrate molar ratio throughout the conifer somatic embryogenesis process.

After fecundation, zygotic embryos of gymnosperms develop out of a non nuclear structure, through a process that can have some variants. Pine zygotic embryos are fertilized in summer time, although full embryo development can happen in two consecutive summers (depending on the species). A structure develops within the archegonium with 16 elongated cells that will become a pre-embryo, which itself may divide and give rise to normal cleavage polyembryogenesis of one or more genotypes when more than one egg is fertilized. In a normal process suspensor cells pushes the embryonic head towards the gametophyte and maturation starts when suspensor cells transfer nutriments to the embryo head from the gametophyte base. Simultaneously, desiccation starts in both zygotic embryo and gametophyte, in such a way that when the embryo has achieved complete maturation, moisture conditions are minimum for it to go into dormancy stage and stay in it until conditions are favorable for germination and normal growth into a full plant. According to the above description, gymnosperm somatic embryogenesis requires the same conditions needed to produce normal mature somatic embryos. It is therefore assumed that requirements needed for induction, proliferation and maturation of somatic embryos are basically the same for all conifers species, with specific variants needed, according to the specie needs, in this case Pinus spp. At the same time, regeneration methods developed for Gymnosperms are basically different from methods developed for Angiosperms.

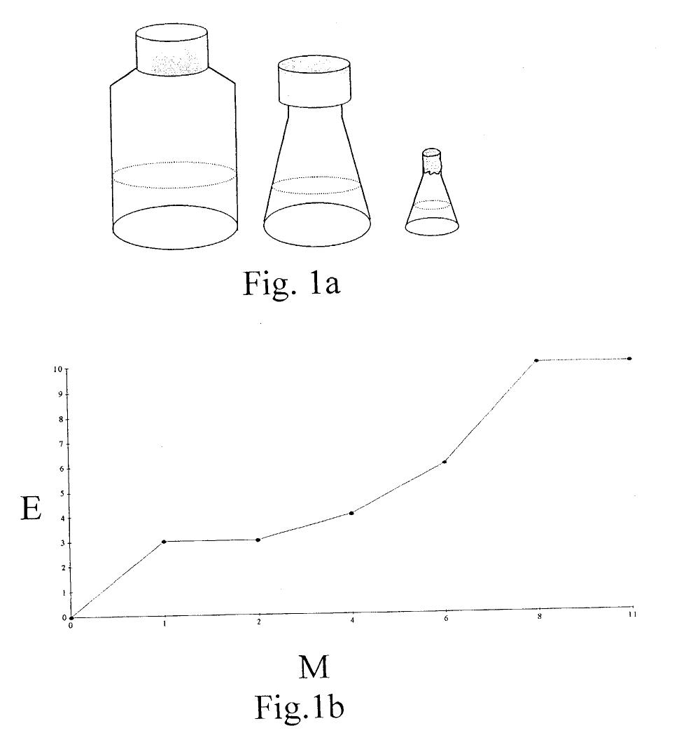

For some Pinaceas trees, as is here the case, the ability to produce mature somatic embryos needs to be maintained for longer periods. However, it is known that continued proliferation of immature embryos leads to loss of maturation capacity and may even lead to loss of proliferation capacity. Under natural latency conditions, which means under the protection of the cone and the female gametophyte, a high percentage of zygotic embryos are able to survive winter time or dry season. Because of this, the effect on proliferation of chilling preservation of immature embryos has been evaluated by exposition time and under 5 different concentrations of plant growth regulators, for Genotype 1. The basic concentration was 2 mg/l 2,4-D and 1 mg/l BA (100%) that experiment was the clue for the best concentration to reinitiate proliferation. During the experiments 500 ml flasks were used, with 100 ml of liquid medium supplemented with 40/60 ammonium to nitrate, without plant growth regulators and with 33 ml of embryogenic suspension (5, 6, 11 and 15 somatic embryos/ml), and then treated by chilling at 4° C. Six full evaluations of reinitiation multiplication time were made, depending on the chilling period, with various concentration of plant growth regulators on the same basal medium. During the 1^{st}, 2^{nd}, and 4^{th} months the same medium was used, with 100% PGR concentration. At 6^{th} month reinitiation time was evaluated for three PGR concentrations: 100, 75 and 50%, and finally, at the 8^{th} and 11^{th} months with only 50%. 5 ml of the somatic embryo chilled suspension were used for each of 4 samples per treatment. The best concentration was found by measuring the size of proliferating immature somatic embryos for each treatment, as well as by counting the number of immature embryos per milliliter at the start and after 10 subcultures. At 11 storage months and after 6 reinitiation evaluation, the immature embryos were able to proliferate. It has been found that storage time and reactivation time have a correlation of 0.94 at 99% confidence level. Longer chilling periods mean longer reactivation periods. For one month of chilling storage, for example, reactivation time was 3 weeks, and for the last evaluation, done after 11 months of chilling, immature embryos needed 11 weeks to start proliferation. Evaluations were only made for an 11 month chilling period, because there were not enough immature embryo samples for further experiments (FIG. 1).

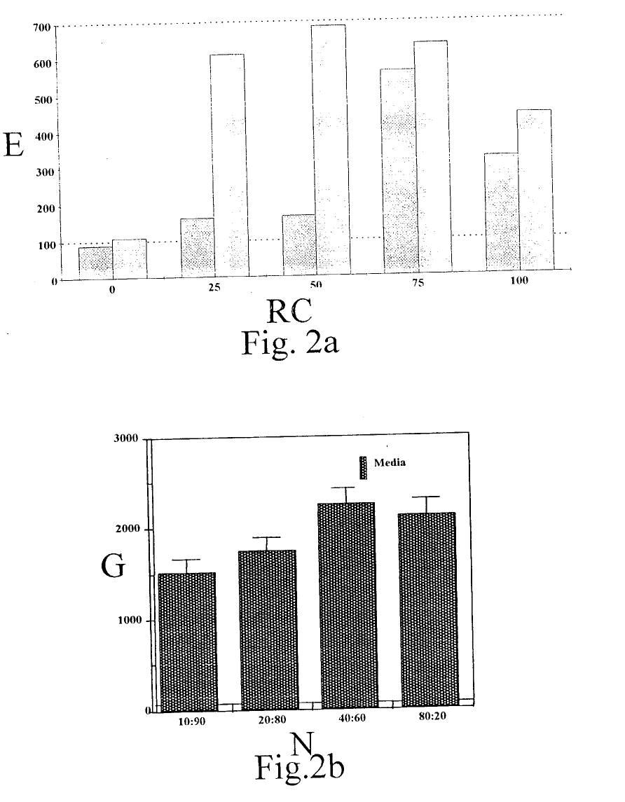

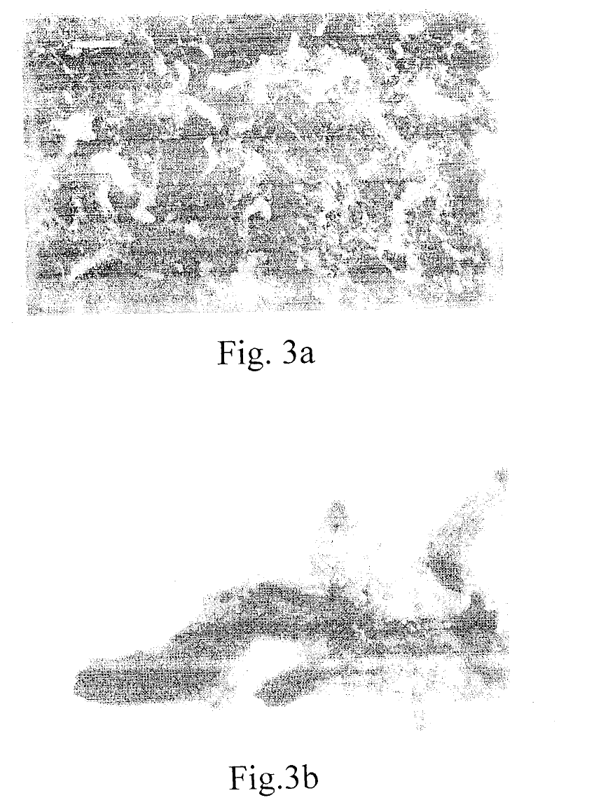

Results show differences on immature embryo multiplication levels due to plant growth regulator concentration, with a positive correlation. For higher PGR concentrations, higher multiplication levels were obtained, however the multiplication period was the same for all treatments. Moreover, it was found that, for the two higher PGR concentrations tested, deformation of immature embryos started after 2 subcultures, and there was no multiplication in any of the treatments with 5 and 6 embryos/ml. The lowest PGR concentration tested (50%) had the slowest multiplication rate (only 50 embryos/ml). However, after 10 subcultures 600 somatic embryos/ml were obtained, and the shape of all of them was perfect. (FIGS. 2a and 3b.).

With respect to PGR concentration, results were as follows: at 100% subculture concentration after reinitiation using embryos chilled for 1, 2 and 4 months, immature embryos quickly lost their embryogenic capacity. At 75%, after 6 months of embryo chilling treatment, a drastic change in embryo shape was observed, until all of them disappeared after 4 subcultures. However, at 50% concentration no embryo morphology change was observed. At 8 months of chilling treatment of immature embryos, proliferation evaluation after 10 subcultures showed that all of them were in very good shape, mainly due to the right quantity of embryos used per subculture and to the lowest concentration of PGR tested (25%). Variance analysis showed significant differences in embryo head size and suspensor cell size, as well as in the full length of immature embryos among treatments, due to PGR, (Table 1). Significant differences were found also through LSD means analysis (Table 2), as was demonstrated also after reinitiation at 11 months of chilling treatment, for a long period of proliferation maintenance, where embryos multiplied very well every 7-15 days (FIG. 3b).

TABLE 1
Variance analysis to evaluate the effect of plant growth regulators
on the size of Genotype 1 immature somatic embryos.

| | | | | |
|---|---|---|---|---|
| SOURCE | FG | MS | F Value | Prob>F |

Embryo head

| | | | | |
|---|---|---|---|---|
| PGR | 4 | 0.0779 | 10.49 | 0.0001** |
| Error | 245 | 0.0074 | | |
| Total | 249 | | | |

Suspensor Cells

| | | | | |
|---|---|---|---|---|
| PGR | 4 | 1.6929 | 8.86 | 0.0001** |
| Error | 245 | 0.1911 | | |
| Total | 249 | | | |

Whole somatic embryo

| | | | | |
|---|---|---|---|---|
| PGR | 4 | 2.3588 | 10.42 | 0.0001** |
| Error | 245 | 0.2264 | | |
| Total | 249 | | | |

**significant level at 0.01

TABLE 2
Significant differences by plant growth regulators in Genotype 1

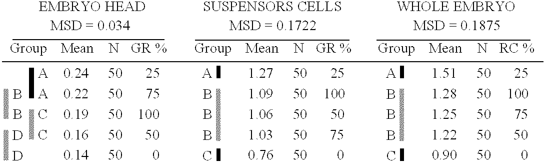

MSD = Minimum significant difference;
z = significant level 0.05

The effect of chilling treatment on proliferation reinitiation were afterwards evaluated for various genotypes. Four 125 ml flasks were used with 45 ml of liquid medium supplemented with 40:60 ammonium to nitrate concentration, without PGR, and adding 10 ml of suspension culture from embrygenic tissue of the eight genotypes to be evaluated (there were approximately 20 immature embryos/ml in each flask's suspension). Reinitiation of proliferation tests were conducted after one month of chilling treatment in culture medium supplemented with 50% PGR concentration used in Genotype 1, and adding 5 ml of chilled embryos in suspension for each sample. Reinduction time and quantity of immature embryos/ml data were registered from all genotypes tested.

Immature embryos multiplied in a medium supplemented with the same ammonium to nitrate 40:60 and PGR concentration as initiation medium for each genotype (Table 3). Differences were found among genotypes in the number of immature embryos/ml and in subculture periods. No data on initiation are shown, only data about the loss of embryogenic capacity when the same initiation medium as in subcultures is used.

TABLE 3
Genotype response to multiplication medium.

| | EMBRYOS/ml | DAYS BEETWEN | EMBRYOS/ml | EMBRYOGENIC |
|---|---|---|---|---|
| GENOTYPE | INITIAL | SUBCULTURES | FINAL | CAPACITY |
| G2 | 25 | 15 | 350 | bad |
| G3 | 35 | 8 | 750 | good |
| G4 | 30 | 8 | 520 | good |
| G5 | 25 | 8 | 545 | good |
| G6 | 29 | 15 | 410 | excellent |
| G7 | 29 | 15 | 485 | excellent |
| G8 | 40 | 15 | 60 | bad |
| G9 | 30 | 8 | 180 | average |

Genotypes G2, G3 and G4 proliferated on liquid medium with 100% PGR concentration; G5, G6, G7 and G8 proliferated in medium with 75% PGR concentration and G9 with 50% concentration.

In chilling treatment at 4° C. and reinitiation period evaluation differences were found in tolerance to chilling treatment and response time by genotype (Table 4). After 1 month of chilling, three genotypes were not able to start multiplication but other genotypes had the same response as Genotype 1. On the other hand two genotypes started proliferation after 6 months.

TABLE 4
Genotype response to chilling treatment.

| | EMBRYOS/ml | TIME | EMBRYOS/ml | EMBRYOGENIC |
|---|---|---|---|---|
| GENOTYPE | INITIAL | TO SUBCULTURE | FINAL | CAPACITY |
| G2 | 20 | — | — | stopped |
| G3 | 20 | 6 months | 600 | good |
| G4 | 20 | 6 months | 550 | good |
| G5 | 20 | 1 month | 520 | good |
| G6 | 20 | 1 month | 400 | excellent |
| G7 | 20 | 1 month | 450 | excellent |
| G8 | 20 | — | — | stopped |
| G9 | 20 | — | — | stopped |

Multiplication after initiation for all genotypes. The quantity of immature embryos for each initial subculture was a key factor. It must be 20-40 embryos/ml, in which case suspension culture is capable of sustaining up to 1000-1500 embryos/ml. Differences were found among genotypes in embryogenic capacity or in multiplication rates. Using the right inoculums, immature embryos of the genotypes tested could be subcultured for 5 to 15 subcultures, depending on the genotype's reinitiation period.

TABLE 5
Genotype multiplication response using
medium with 25% of PGR concentration.

| | EMBRYOS/ml | TIME | EMBRYOS/ml | EMBRYOGENIC |
|---|---|---|---|---|
| GENOTYPE | INITIAL | TO SUBCULTURE | FINAL | CAPACITY |
| G2 | 20-40 | 15 | 500-1000 | excellent |
| G3 | 20-40 | 15 | 750-1500 | excellent |
| G4 | 20-40 | 15 | 520-1000 | excellent |
| G5 | 20-40 | 15 | 545-1300 | excellent |
| G6 | 20-40 | 15 | 410-800 | excellent |
| G7 | 20-40 | 15 | 500-1000 | excellent |
| G9* | 30-40 | 15 | 600-1200 | excellent |

*Proliferation without chilling treatment at 4° C.

The above results show that the proliferation rates in suspension cultures are affected by the PGR concentration and by each genotype's embryogenic capacity. This knowledge enables the proliferation of each genotype for one year without any loss of embryogenic or proliferation capacity and without causing deformation of immature embryos (Table 5).

It must be emphasized that the 80:20 ammonium to nitrate ratio promotes a high proliferation rate on a solid medium (FIG. 2b), as well as in suspension cultures, with better response if the liquid medium is supplemented with the lowest PGR concentration and carbon source, which can be maltose or sucrose.

On the other hand, it was important to device an intermediate step between proliferation in liquid medium and the maturation process in order to prepare the immature embryo to respond to the maturation promoter. This capacity is acquired by immature embryos that have proliferated in medium supplemented with 10:90 or 20:80 ammonium to nitrate ratio (FIG. 2b), 3% maltose and no PGR. For best results it is necessary to completely eliminate the proliferation medium used, washing also two times with distilled water and transfer the immature embryos in liquid medium again. The embryo suspension becomes dark after two subcultures, which means that immature embryos are ready to be transferred to solid medium supplemented with an adsorbent in order to completely stop proliferation and to allow embryo development.

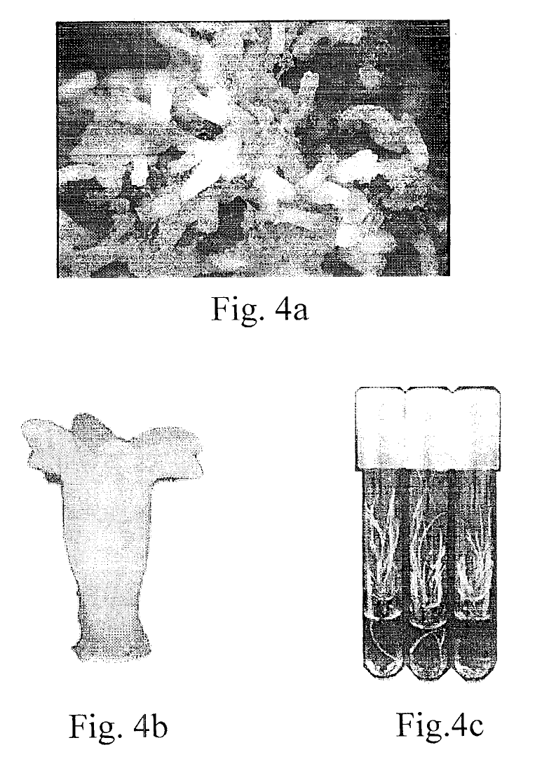

The maturation process which includes maturation pretreatment was done in a medium supplemented with 1% activated charcoal, 10:90 ammonium to nitrate ratio, 3% maltose and the gelling agent was gellan gum 0.35%, the quantity of washed embryos must be between 150-200 mg per sample, dispersed as a thin layer. The washing step was performed three times, in order to eliminate the substances that induce proliferation. Then the immature embryos must be transferred to a solid medium, any excess liquid must be eliminated and, if necessary, samples must be exposed to sterile airflow chamber in order to dry both the embryos and the medium. The treatment must be applied for as long as needed in order to stop proliferation (2-8 weeks). In this way response was optimized and embryos preserved by chilling treatment at 4° C. from all genotypes were cultured in suspension (FIG. 4a and FIG. 4b) and able to mature.

The maturation process, is initiated by the interaction between nitrogen and a carbon source of the right type, with a high concentration of ABA, and at least one desiccating agent. Abscisic acid must be added in high concentration at the onset of maturation in order to obtain high quality embryos and prevent precocious germination. For most conifers, concentration must fluctuate between 16 μM and 24 μM (±) ABA. However, the Pinus genera requires between 60 to 100 μM of ABA. Usually, the most efficient source of ABA is a razemic mixture (±), since it has given best results. For this test only 80 μM ABA were used, since previous tests with 20, 35 and 60 μM had effect on the genotypes mentioned above. Orthodox zygotic embryos require a desiccation period to start normal germination. Also, gymnosperm somatic embryos have to be treated in a medium supplemented with a high concentration of a desiccant substance, in order to avoid precocious germination. Different types of sugar substances have been used as desiccating agents, in a concentration of up 6 to 9%. However, for this new method we used an inert compound that is not metabolized by the embryo cell as is the case with sugars, a compound that also generates a medium dry condition, which is the key to promote accumulation of storage compounds within the cells. It is known that when this desiccant compound is used in a wrong concentration, the embryogenic tissue either proliferates or becomes dehydrated (Table 6). The most useful compound is PEG 4000 MW, since it generates a desirable viscosity. Gellan gum can also be used to produce the same medium condition. This compound is highly important for medium quality, since it is a polymer that does not react with any other compound in the medium, being almost an inert substance. Its only function is to provide support for the medium, in order to have the physical characteristics required by a medium for in vitro culture. Another physical requirement is to avoid liquefaction and that it is not absorbed by the plant. Its concentration determines the availability of water molecules that can be absorbed by cells.

The quantity of embryos per sample exposed to the maturation medium was another key factor for the optimization of this method for producing somatic embryos. This knowledge was obtained from the genotypes that have the lowest proliferation rates, when a small quantity of immature embryos was exposed to maturation medium as a thin layer which in turn produced mature somatic embryos. It was also found that, for genotypes with high proliferation rates, response to maturation medium was practically nil.

Two experiments were performed to test the effect of one or two desiccant compounds on the medium, using immature somatic embryos from genotypes chilled at 4° C. It has been reported that the optimum concentration of gellan gum is 1% without PEG, but for a different ammonium to nitrate molar ratio and other compounds. In order to test this, an experiment was carried out using a maturation medium supplemented with 40:60 ammonium to nitrate ratio, 3% sucrose, ABA 35 μM, with or without PEG 4000 7.5% and with various concentrations of gellan gum (Table 6); where embrygenic tissue becomes fully dehydrated in medium with up to 0.7% concentration, but where at 0.35% concentration proliferation continues. On the basis of these results, a medium supplemented with 10:90 ammonium to nitrate ratio, ABA 80 μM, maltose 3%, and 0.55% gellan gum was tested in order to obtain dry conditions in a medium without PEG 4000. A high rate of mature somatic embryos was obtained (FIG. 4a and FIG. 4b), which proves that it is possible to preserve embryogenic capacity, since mature somatic embryos can be obtained after chilling treatment at 4° C.

TABLE 6
PEG and gellan gum effect on embryo maturation treatment in
a medium supplemented with 40:60 ammonium to nitrate ratio,
ABA 35 μM and sucrose 3%, using more than 400 mg of
embryogenic tissue per sample.

| Gellan gum ± PEG 7.5% | | Mature somatic |
|---|---|---|
| Concentration | Response | embryos/ sample |
| 0.35% | Proliferation | 0 |
| 0.55%* | Maturation | 50 ± 10 |
| 0.7% | Desiccated tissue | 0 |
| 1.0% | Desiccated tissue | 0 |
| 1.4% | Desiccated tissue | 0 |

*This treatment was given by maturation medium supplemented with 10:90 ammonium to nitrate ratio, maltose 3%, ABA 80 μM without PEG and 150-200 mg of immature embryos per sample.

This invention has various important and distinctive characteristics. It is the first time that a method for preserving somatic embryos other than cryopreservation is reported, that makes the regeneration of conifer mature somatic embryos possible (FIG. 4c).

This method allows preservation of conifer somatic embryos in suspension for a period as long as 11 months without loss of proliferation capacity. It was demonstrated that embryogenic capacity depends on genotype and on PGR concentration. However, for the best genotypes, proliferation can be reinitiated when needed, and at the same time the embryo suspension can be chilled again at a 4° C. for another period.

Is the first time that suspension cultures with 10:90 ammonium to nitrate are used to reduce the embryogenic proliferation as a previous step for maturation pretreatment.

We have also proved that for best results during the maturation method, proliferation ratio must be reduced by using a medium supplemented with 10:90 ammonium to nitrate, immature embryos must be washed with distillated sterile water, drawn onto filter paper, and from 150 to 200 mg of embryogenic tissue per sample, distributed as a thin layer. Pretreatment must also be used to start somatic embryo development in the above described medium, and somatic embryo treatment onto maturation medium supplemented with the relation 10:90 ammonium to nitrate, 3% maltose, ABA 80 μM and 0.55% of gellan gum. The method succeeded in maturating somatic embryos obtained from suspension cultures reinitiated from chilling preservation of embryos at 4° C., as the only way to preserve embryogenic capacity of immature embryos which proliferate in liquid medium.

This methodology will be useful in preserving embryogenic capacity in suspension cultures of genotypes during evaluation, cultures that can also be cryopreserved as a tool for breeding programs or genetic transformation, among other studies that have to start after a successful plant regeneration protocol has been developed.

Key Words

The technique named as “suspension culture” is defined as a semi continuous culturing method done in Erlenmeyer flasks, where immature embryos proliferate exponentially, which means cultures must be restarted periodically by picking up the right amount of liquid suspension of immature somatic embryos from the previous culture, and transferring the suspension to new liquid sterile medium. The rest of the suspension can be used or preserved, or it can be eliminated.

### REFERENCES

Aitken-Christie, J. and Connett, M. 1992. Micropropagation of Forest Trees. In: Transplant Production System. Kurata, K. y T. Kozai (eds.) Kluwer Academic Publisher, The Netherlands. Pp.: 163-194.

Attree, S. 1999. Increasing levels of growth regulator and/or water stress during embryo development. WO9963805A2 patent. Dec. 16, 1999.

Attree, S. and L. C. Fowke. 1999. Maturation, desiccation and encapsulation of gymnosperm somatic embryos. U.S. Pat. No. 0,598,5667 Patent. Nov. 16, 1999.

Attree, S. M. and Fowke, L. C. 1993. Embryogeny of gymnosperms: advances in synthetic seed technology of conifer. Plant Cell Tiss. Org. Cult. 35: 1-35.

Attree, S. M.; Dunstan, D. I. and Fowke, L. C. 1989. Plant regeneration from embryogenic protoplasts of white spruce (Picea glauca). Bio/Technol. 7. 1060-1062.

Attree, S. M.; Pomeroy, M. K. and Fowke, L. C. 1994. Production of vigorous, desiccation, tolerant white spruce (Picea glauca (Moench,) Voss.) synthetic seed in bioreactor. Plant Cell Rep. 13: 601-606.

Becwar, M. R.; Chesick, E. E.; Handley, III, L. W. and M. R. Rutter. 1995. Method for regeneration of coniferous plants by somatic embryogenesisa. U.S. Pat. No. 0,541,3930 patent. May 9, 1995.

Coke, J. E. 1996. Basal nutrient medium for in vitro cultures of loblolly pines. U.S. Pat. No. 0,553,4434 patent. Jul. 9, 1996.

DeVerno, L. L.; Park, Y. S.; Bonga, J. M., Barret, J. D. and Simmpson. C. 1999. Somaclonal variation in cryopreserved embryogenic clones of white spruce (Picea glauca (Moench) Voss.). Plant Cell Rep. 18 (11): 948-953.

Dunstan, D. I.; Bethune, T. D. and Bock, C. A.1993. Somatic embryo maturation from long-term suspension cultures of white spruce (Picea glauca).In vitro Cell. Dev. Biol. 29:109-112.

Eastman, P. A. K.; Webster, F. B.; Pitel, J. A. and Roberts, D. R. 1991. Evaluation of somaclonal variation during somatic embryogenesis of interior spruce (Picea glauca engelmannii complex) using culture morphology and isozyme analysis. Plant Cell Rep. 10: 425-430.

Finer, J. J.; Kriebel, H. B. and Becwar, M. R. 1989. Initiation of embryogenic callus and suspension cultures of eastern white pine (Pinus strobus L.) Plant Cell Reports 8: 203-206.

Garin, E.; Isabel, N. and Plourde, A. 1998. Screening of large numbers of seed families of Pinus strobus L. For somatic embryogenesis from immature and mature zygotic embryos. Plant Cell Rep. 18: 37-43.

George, E. F. 1993. The Technology. In: Plant propagation by tissue culture. Part1. Exegetics Ltd. (eds.) Butler & Tanner Ltd. England. Pp. 470.

Gupta, P. K. 1996. Method for reproducing conifers by somatic embryogenesis using a maltose enriched maintenance medium. U.S. Pat. No. 0,556,3061 patent. Oct. 8, 1996.

Gupta, P. K. and Durzan, D. J. 1987a. Biotechnology of somatic polyembryogenesis and plantlet regeneration in loblolly pine. Bio/Technol. 5: 147-151.

Gupta, P. K. and Durzan, D. J. 1987b. Somatic embryos from protoplast of loblolly pine proembryonal cells. Bio/Technol. 5: 710-712.

Gupta, P. K. and G. S. Pullman. 1991. High concentration enrichment of conifer embryonal cells. U.S. Pat. No. 0,504,1382 patent. Aug. 20, 1991.

Gupta, P. K. and G. S. Pullman. 1991. Method for reproducing conifers by somatic embryogenesis using abscisic acid and osmotic potential variation. U.S. Pat. No. 5,036,007 patent. Jul. 30, 1991.

Gupta, P. K. and G. S. Pullman. 1993. Method for reproducing conifers by somatic embryogenesis using stepwise hormone adjustment. U.S. Pat. No. 0,523,6841 patent. Aug. 17, 1993.

Gupta, P. K.; Pullman, G.; Timmis, R.; Kreitinger, M.; Carlson, W. C.; Grob, J. and Welty, E. 1993. Forestry in the 21^{st}. century. The biotechnology of somatic embryogenesis. Bio/Technol. 11: 454-459.

Hakman, I. and von Arnold, S.1985. Plantlet regeneration through somatic embyogenesis in Picea abies (Norway spruce). J. Plant Physiol. 121: 579-587.

Handley III, L. W. and Godbey, A. P. 1996. Embryogenic coniferous liquid suspension cultures. U.S. Pat. No. 0,544,91090 patent. Feb. 13, 1996.

Handley, III, L. W. 1998. Method for regeneration of coniferous plants by somatic embryogenesis. U.S. Pat. No. 0,573,1203 patent. Mar. 24, 1998.

Handley, III, L. W. 1999. Method for regeneration of coniferous plants by somatic embryogenesis in culture media containing abscisic acid. U.S. Pat. No. 0,585,6191 patent. Jan. 5, 1999.

Hogberg, K.-A.; Ekberg, I.; Norell, L. and von Arnold, S. 1998. Integration of somatic embryogenesis in a tree breeding programme: a case study with Picea abies. Can. J. For. Res. 28: 1536-1545.

Joy, R. W.; Parkash, P. K. and Thorpe, T. A.1991. Long term storage of somatic embryogenic white spruce tissue at ambient temperature. Plant Cell Tiss. Org. Cult. 25: 53-60.

Klimaszewska, K. and Smith, D. 1997. Maturation of somatic embryos of Pinus strobus is promoted by high concentration of gellan gum. Physiol Plant. 100: 949-957.

Laine, E. and David, A. 1990. Somatic embryogenesis in immature embryos and protoplasts of Pinus caribaea. Plant Science 69: 215-224.

Laine, E.; Bade, P. and David, A. 1992. Recovery of plants from cryopreserved embryogenic cell suspensions of Pinus caribaea. Plant Cell Rep. 11: 295-298.

Lelu, M. A.; Bastien, C.; Drugeault, A.; Gouez, M L. and Klimaszewska, K. 1999. Somatic embryogenesis and plantlet development in Pinus sylvestris and Pinus pinaster on medium with and without growth regulators. Physiol Plant. 105: 719-728.

McVaugh, R. 1992. Gymnosperms and Pteridophytes. In: Flora Novogaliciana. Anderson, W. R. (ed.) University of Michigan. United States of America. Vol. 17: 1-119.

Pullman, G. S. and P. K. Gupta. 1991. Method for reproducing coniferous plants by somatic embryogenesis using adsorbent materials in the development stage media. U.S. Pat. No. 0,503,4326 patent. Jul. 23, 1991.

Pullman, G. S. and P. K. Gupta. 1994. Method for reproducing conifers by somatic embryogenesis using mixed growth hormones for embryo culture. U.S. Pat. No. 0,529,4549 patent. Mar. 15, 1994.

Ramírez-Serrano, C. 1996. Embriogénesis somática en pino pinõnero (Pinus maximartinezii Rsedowski). MS Thesis. CIATEJ-UDEG. Guadalajara Jal. Mexico. 72 pp.

Ramírez-Serrano, C.; Bozkhob, P.; Ekberg, I and von Arnold, S.1999a. Potential of somatic embryogenesis for a wide range of genotypes in Scots pine (Pinus sylvestris L.). In:

Program-Abstracts XIX Congress Scandinavian Society of Plant Physiology. Joensuu Finland, Jun. 21-23, 1999. Pp. 103.

Ramírez-Serrano, C.; Bozkhob, P.; Ekberg, I. and von Arnold, S.1999b. Inter and Intra family effects in somatic embryogenesis of scots pine. In: Abstracts Forest Biotechnology'99. A join Meeting of: The International Wood Biotechnology Symposium and IUFRO Working Party 2.40-06 Molecular Genetics of Trees. Kable College, University of Oxford, Oxford, United Kingdom. Jul. 11-16, 1999. Poster 7.

Rutter, M. R.; Handley, III, L. W. and M. R Becwar. 1998. Method for regeneration of coniferous plants by somatic embryogenesis employing polyethilene glycol. U.S. Pat. No. 0,573,1204 patent. Mar. 24, 1998.

Tautorus, T. E.; Fowke, L. C. and Dunstan, D. I.1991. Somatic embryogenesis in conifers. Can. J. Bot. 69: 1873-1899.

Uddin, M R. 1993. Somatic embryogenesis in gymnosperms. U.S. Pat. No. 0,518,7092 patent. Feb. 16, 1993.

von Arnold, S.1987. Improved efficiency of somatic embryogenesis in mature embryos of Picea abies (L.) Karst. J. Plant Physiol.128: 233-244.

Webster, F. B.; Roberts, D. R.; McInnis, S. M. and Sutton, B. C. S. 1990. Propagation of interior spruce by somatic embryogenesis. Can. J. For. Res. 20: 1759-1765.

1. A method for producing mature somatic embryos of Pinaceae trees which is based in performing refrigeration and suspension cultures to maintain embryogenic capacity; which comprises the preservation of immature somatic embryos by refrigeration, reinduction of proliferation achieved into a period varying between 1 to 6 months, the establishment of proliferation and maintenance, the treatment to reduce the proliferation rate, prematuration treatment and maturation of somatic embryos.

2. A method in accordance with claim 1, wherein said preservation technique of immature somatic embryos is performed by means of one culture medium with enough amount of nutriments in preferred physical state, favorable temperature, preservation medium compounds and the adequate quantity of immature somatic embryos per ml.

3. A method in accordance with the claim 2, wherein said one culture medium in preferred physical state is liquid medium.

4. A method in accordance with the claim 2, wherein said favorable temperature is ranging between 0° to 10° C.

5. A method in accordance with the claim 2, wherein said preservation medium compounds are a carbon source, an organic nitrogen source, and an amino acid complex source.

6. A method in accordance with the claim 5, wherein said a carbon source is sucrose, maltose, glucose.

7. A method in accordance with the claim 6, wherein the sucrose, maltose or glucose concentration is in the range from 1 to 10%.

8. A method in accordance with the claim 5, wherein said organic nitrogen source is glutamine, arginine.

9. A method in accordance with the claim 8, wherein said the concentration of glutamine or arginine is in the range between 50 to 600 mg/l.

10. A method in accordance with the claim 5, wherein said amino acid complex source is casein hydrolyzed.

11. A method in accordance with the claim 10, wherein the concentration of casein hydrolyzed is in the range between 50 to 2000 mg/l.

12. A method in accordance with claim 2, wherein the adequate quantity of immature somatic embryos per ml is in the range between 5 to 2000.

13. A method in accordance with claim 1, wherein reinduction of embryogenic tissue is from good to excellent after preservation by refrigeration, which is achieved throughout of treatment of the adequate quantity of immature somatic embryos in liquid medium or in diverse liquid media, various concentrations of the mixture of PGR but at the same (2:1) ratio, plus carbon source.

14. A method in accordance with the claim 13, wherein said adequate quantity of immature somatic embryos per ml varying between 5 to 1000.

15. A method in accordance with claim 13, wherein said liquid medium for reinduction, are supplemented with ammonium to nitrate molar ratio in the range between 99:01 to 01:99.

16. A method in accordance with the claim 13, wherein the mixture of PGR is the combination ranging from 0 to 10 mg/l of auxin and from 0 to 5 mg/l of cytokinin

17. A method in accordance with claim 16, wherein a concentration of PGR varying in each used medium in the range between 0 to 200%.

18. A method in accordance with claim 613 wherein said carbon source is either sucrose or maltose with a concentration in the range between 1 and 10%.

19. A method in accordance with claim 1, wherein said establishment of proliferation and its maintenance is excellent, by means of suspension cultures of the adequate quantity of immature somatic embryos per ml, within a modified media with a different ammonium to nitrate molar ratio and the best PGR concentration, during a period varying between 1 to 24 months.

20. A method in accordance with claim 19, wherein said enough quantity of immature somatic embryos is ranging between 5 to 1000 per ml.

21. A method in accordance whit claim 19, wherein each modified medium is supplemented with a relation of ammonium to nitrate ranging between 99:01 to 01:99.

22. A method in accordance with the claim 19, wherein said the best concentration of PGR is ranging between 0 to 100%.

23. A method in accordance with claim 1, wherein treatment to reduce proliferation rates is in order to encourage the immature somatic embryos to the activity of a maturation promoter, by means of suspension cultures with a specialized medium that contains a low ammonium to nitrate molar ratio, a carbon source and lacking of PGR, for a period from 1 to 12 weeks.

24. A method in accordance with the claim 23, wherein said treatment to reduce proliferation rates is including to eliminate the previous proliferation medium, to wash with both sterile distillates water or liquid medium between 1 to 5 times, and then resuspending in specialized medium.

25. A method in accordance with the claim 16, wherein said low ammonium to nitrate ratio ranges between 01:99 and 40:60.

26. A method in accordance with claim 16, wherein said carbon source is sucrose or maltose.

27. A method in accordance with claim 19, wherein the concentration of sucrose or maltose is between 1 and 10%.

28. A method in accordance with claim 1, wherein pretreatment of maturation is to stop proliferation and to start the development of the immature somatic embryos from suspension cultures, by means of a modified solid medium with an ammonium to nitrate ratio ranging between 01:99 to 40:60, a carbon source, a chemical adsorbent, and no PGR, for a period ranging from 1 to 12 weeks.

29. A method in accordance with claim 28, wherein the prematuration treatment is including the washing of immature somatic embryos with both sterile distillates water or a liquid medium between 1 to 5 times previous to expose them to the modified solid medium.

30. A method in accordance with claim 28, wherein the pretreatment of maturation includes dispersing between 50 to 1000 mg of immature somatic embryos as a thin layer on filter paper.

31. A method in accordance with claim 28, wherein the carbon source is sucrose or maltose

32. A method in accordance with claim 31, wherein said sucrose or maltose is present in the medium in a concentration that ranges between 1 and 10%.

33. A method accordance with claim 28, wherein said chemical adsorbent is activated charcoal.

34. A method in accordance with claim 1, wherein said maturation is for the immature somatic embryos that had stopped proliferation; by means of the treatment in a special solid medium supplemented with a low ammonium to nitrate molar ratio, carbon source, a maturation promoter and a desiccant compound.

35. A method, in accordance with claim 34, wherein said low ammonium to nitrate ratio ranges from 01:99 to 40:60.

36. A method, in accordance with claim 34, wherein said carbon source is sucrose or maltose.

37. A method, in accordance with claim 36, wherein the sucrose or maltose concentration ranges from 1 to 10%.

38. A method, in accordance with claim 34, wherein said a maturation promoter is ABA or analogous.

39. A method, in accordance with claim 38, wherein the concentration of ABA or analogues ranges from 50 to 120 μM.

40. A method, in accordance with claim 34, wherein said desiccant compound is gellan gum.

41. A method, in accordance with claim 40, wherein gellan gum concentration ranges from 0.3 to 1.2%.

42. A pinaceae mature somatic embryo which is capable to germinate and growths as seedling, characterized by having the ancestral immature somatic embryos resistance to refrigeration treatment, to perform from good to excellent reinitiation, to maintain an excellent proliferation, and to show capacity to reduce proliferation rate, these steps were performed by means of suspension cultures; besides to tolerate a treatment to stop proliferation, and finally achieving maturation in a medium supplemented with a low ammonium to nitrate molar ratio.

43. A pinaceae mature somatic embryo, in accordance with claim 42, characterized by being analogous to pinacea zygotic embryo.

44. A pinaceae mature somatic embryo in accordance with claim 42, wherein said mature somatic embryo is from the genus Pinus.

45. A mature somatic embryo, in accordance with claim 44, wherein said mature somatic embryo is to all embryos come from the genus Pinus.

46. A method, in accordance with claim 42, wherein said embryos are developed in a time varying from 1 to 15 weeks.

47. A method, in accordance with claim 42, wherein said embryos are developed in a time varying from 3 to 10 weeks.

48. A method, in accordance with claim 42, wherein said embryos are developed in a time varying from 5 to 8 weeks.

49. A method in accordance with claim 42, wherein all of said mature somatic embryos originated from genotypes were preserved by refrigeration for a period ranging from 1 to 36 months.

50. A method in accordance with claim 42, wherein all of said mature somatic embryos originated from genotypes which were preserved by refrigeration and/or reinitiated and performed from good to excellent proliferation by means of suspension cultures for a period ranging from 1 to 36 months.

51. A method in accordance with claim 42, wherein all of said mature somatic embryos originated from genotypes that were preserved by refrigeration and/or performed from good to excellent establishment and its maintenance by means of suspension cultures for a period ranging from 1 to 36 months.

52. A method in accordance with claim 42, wherein all of said mature somatic embryos originated from genotypes that had a treatment to reduce proliferation rates by means of suspension cultures in a period varying from 1 to 12 weeks.

53. A method in accordance with claim 42, wherein all of said mature somatic embryos originated from genotypes that reduced or no shown proliferation by means of washing with both sterile distillated water or liquid medium followed by pre maturation treatment.

54. A method in accordance with claim 42, wherein all of said mature somatic embryos originated from genotypes were preserved by refrigeration, and to treat in an amount varying from 50 to 1000 mg of the immature somatic embryos as thin layer in a maturation medium with low ammonium to nitrate molar ratio and a desiccant compound.
